(19) United States
(12) Patent Application Publication
(10) Pub. No.: US 2022/0280234 A1
(43) Pub. Date: Sep. 8, 2022

(54) OPTICAL OUTPUT CONTROL DEVICE AND LASER DEVICE HAVING SAME

(71) Applicant: LUTRONIC CORPORATION, Goyang-si, Gyeonggi-do (KR)

(72) Inventor: Hee Chul Lee, Goyang-si, Gyeonggi-do (KR)

(21) Appl. No.: 17/597,848

(22) PCT Filed: Aug. 28, 2020
(86) PCT No.: PCT/KR2020/011539

§ 371 Date: Jan. 26, 2022

(30) Foreign Application Priority Data
Aug. 29, 2019 (KR) 10-2019-0106639

(51) Int. Cl. A61B 18/20

(52) U.S. Cl. CPC A61B 18/20; A61B 2018/00702

(57) ABSTRACT

According to an embodiment of the present disclosure, an optical output control device includes: a wavelength-selective reflector configured to reflect light in a predetermined wavelength band and incident at a reference angle θ_{c}; and a driving unit configured to rotate the wavelength-selective reflector to adjust an angle of incidence of light incident on the wavelength-selective reflector.

### TECHNICAL FIELD

Embodiments of the present disclosure relate to an optical output control device and a laser device including the optical output control device.

### BACKGROUND ART

Laser beams are used in various fields for industrial, medical, and military purposes. In particular, medical lasers are widely used in surgeries, internal medicine, ophthalmology, dermatology, dentistry, and the like because they allow concentration of a preset amount of energy at a local site and enable non-invasive treatment.

Medical lasers are required to maintain a proper output level for obtaining therapeutic effects.

### DESCRIPTION OF EMBODIMENTS

### Technical Problem

Provided are an optical output control device and a laser device having a stable optical output level by using the optical output control device.

### Solution to Problem

According to an aspect, an optical output control device includes: a wavelength-selective reflector configured to reflect light in a predetermined wavelength band and incident at a reference angle θ_{c}; and a driving unit configured to rotate the wavelength-selective reflector to adjust an angle of incidence of light incident on the wavelength-selective reflector.

The optical output control device may further include: a photodetector configured to sense an amount of light reflected from the wavelength-selective reflector or passing through the wavelength-selective reflector; and a processor configured to generate, based on the amount of light sensed by the photodetector, a control signal to be transmitted to the driving unit.

The processor may be further configured to predict an output value from the amount of sensed light, and set a rotation angle for the wavelength-selective reflector by comparing the predicted output value with a set target value.

The wavelength-selective reflector may include: a transparent member having an entrance surface and an exit surface, which are opposite each other; and a wavelength-selective coating layer formed on the entrance surface to reflect light in the predetermined wavelength band.

An anti-reflection coating layer may be formed on the exit surface.

The optical output control device may further include an optical path adjuster arranged on a traveling path of light that passed through the wavelength-selective reflector and configured to be rotated for adjusting the traveling path.

The optical path adjuster may be further configured to adjust the traveling path of light that passed through the wavelength-selective reflector to be aligned with a traveling path of the light when the light is incident on the wavelength-selective reflector.

According to an aspect, a laser device includes: a light source unit including a laser medium, a first mirror and a second mirror which are arranged with the laser medium therebetween, and an excitation light source configured to supply light to the laser medium, the light source unit being configured to generate light in a predetermined wavelength band; a wavelength-selective reflector arranged on a path of light output from the light source unit and configured to reflect light in the predetermined wavelength band and incident at a reference angle θ_{c}; a photodetector configured to sense an amount of light reflected from the wavelength-selective reflector or passing through the wavelength-selective reflector; a driving unit configured to rotate the wavelength-selective reflector to adjust an angle of incidence of light incident on the wavelength-selective reflector; and a processor configured to generate a control signal to be transmitted to the driving unit.

The wavelength-selective reflector may be arranged such that an incident angle θᵢ at which light generated from the light source unit is incident may be different from the reference angle θ_{c}.

The processor may be further configured to generate the control signal based on the amount of light sensed by the photodetector.

The photodetector may be arranged to sense the amount of light reflected from the wavelength-selective reflector.

The processor may be further configured to predict an output value from the amount of reflected light, and set a rotation angle for the wavelength-selective reflector by comparing the predicted output value with a set target value.

The processor may be further configured to set a rotation angle for the wavelength-selective reflector by comparing the amount of reflected light with a set reference value.

The wavelength-selective reflector may include: a transparent member having an entrance surface and an exit surface, which are opposite each other; and a wavelength-selective coating layer formed on the entrance surface to reflect light in the predetermined wavelength band.

The laser device may further include an optical path adjuster arranged on a traveling path of light that passed through the wavelength-selective reflector and configured to be rotated for adjusting the traveling path.

The optical path adjuster may be further configured to adjust the traveling path of light that passed through the wavelength-selective reflector to be aligned with a traveling path of the light when the light is incident on the wavelength-selective reflector.

The optical path adjuster may be arranged symmetrically to the wavelength-selective reflector with respect to a plane perpendicular to the traveling path of light that passed through the wavelength-selective reflector.

The optical path adjuster may include a material having a refractive index equal to a refractive index of the transparent member and may have a thickness equal to a thickness of the transparent member.

The driving unit may be further configured to rotate the optical path adjuster in conjunction with rotation of the wavelength-selective reflector such that the optical path adjuster may be rotated by a rotation angle of the wavelength-selective reflector in a direction opposite to a rotation direction of the wavelength-selective reflector.

### Advantageous Effects of Disclosure

The optical output control device may control optical output by using a total-reflection coating layer without using the polarization characteristics of incident light.

The laser device including the optical output control device may have uniform optical output regardless of the polarization state of laser light.

### BRIEF DESCRIPTION OF DRAWINGS

FIG. 1 is a flowchart conceptually illustrating operations of an optical output control device according to an embodiment.

FIG. 2 illustrates a schematic structure of an optical output control device according to an embodiment.

FIGS. 3A and 3B illustrate a detailed structure of a wavelength-selective reflector of the optical output control device shown in FIG. 2 and an optical path along which the amount of light transmission varies according to the angle of incidence of light.

FIG. 4 is a graph illustrating an example in which the amount of light transmission varies according to the angle of incidence on the wavelength-selective reflector of the optical output control device shown in FIG. 2.

FIG. 5 illustrates a schematic structure of an optical output control device according to another embodiment.

FIG. 6 illustrates a schematic structure of an optical output control device according to another example embodiment.

FIG. 7 illustrates a schematic structure of a laser device according to an embodiment.

FIG. 8 is a flowchart conceptually illustrating operations in which the laser device shown in FIG. 7 emits controlled output light.

FIGS. 9 and 10 are example flowcharts illustrating more detailed operations of controlling output light in the laser device shown in FIG. 7.

### MODE OF DISCLOSURE

The present disclosure may have various different forms and various embodiments, and specific embodiments are illustrated in the accompanying drawings and are described herein in detail. Effects and features of the present disclosure, and methods of achieving the effects and features will become apparent with reference to the accompanying drawings and the embodiments described below in detail. However, the present disclosure is not limited to the embodiments described below and may be implemented in various forms.

Hereinafter, the embodiments will be described with reference to the accompanying drawings. In the drawings, like reference numerals denote like elements, and overlapping descriptions thereof will be omitted.

In the following descriptions of the embodiments, although terms such as “first” and “second” are used to describe various elements, these elements should not be limited by these terms. These terms are only used to distinguish one element from another element.

In the following descriptions of the embodiments, the terms of a singular form may include plural forms unless referred to the contrary.

In the following descriptions of the embodiments, the meaning of “include,” “comprise,” “including,” or “comprising” specifies a property or an element, but does not exclude other properties or elements.

In the descriptions of the embodiments, when a region or an element is referred to as being “above” or “on” another region or element, it can be directly on the other region or element, or intervening regions or elements may also be present.

In the drawings, the sizes of elements may be exaggerated for clarity. For example, in the drawings, the size or thickness of each element may be arbitrarily shown for illustrative purposes, and thus the present disclosure should not be construed as being limited thereto.

The order of processes explained in one embodiment may be changed in a modification of the embodiment or another embodiment. For example, two processes sequentially explained may be performed substantially at the same time or in the reverse of the explained order.

In the descriptions of the embodiments, when a region, an element, or the like is referred to as being “connected to,” another region or element, it may be directly connected to the other region or element or may be indirectly connected to the other region or element through intervening regions or elements.

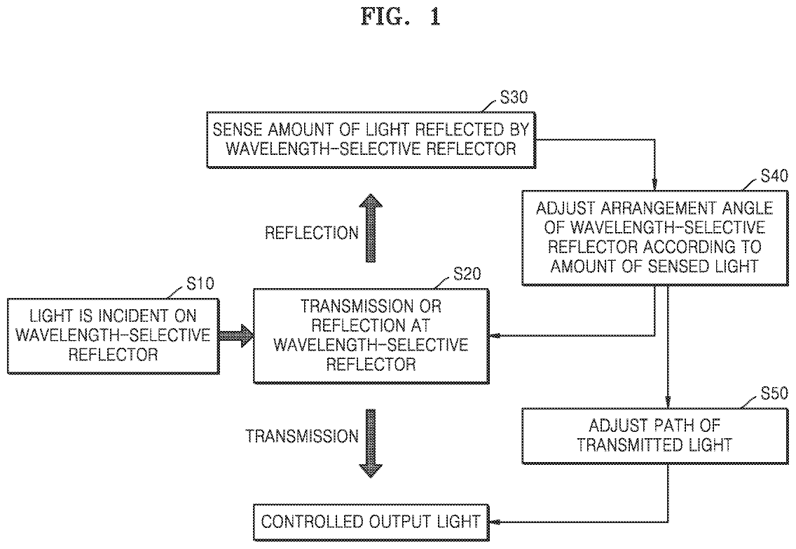

FIG. 1 is a flowchart conceptually illustrating operations of adjusting optical output in an optical output control device according to an embodiment.

A wavelength-selective reflector is an optical member configured to reflect light in a predetermined wavelength band and incident at a predetermined reference angle θ_{c}. When light is incident on the wavelength-selective reflector (S10), a portion of the light satisfying reflection conditions is reflected, and the remaining portion of the light passes through the wavelength-selective reflector (S20).

In this manner, the amount of reflected light and the amount of transmitted light vary depending on the angle of incidence on the wavelength-selective reflector. Because the angle of incidence of light on the wavelength-selective reflector is determined by the arrangement angle of the wavelength-selective reflector with respect to the incident light, the amount of light transmission through the wavelength-selective reflector may be adjusted by sensing the amount of light reflected by the wavelength-selective reflector (S30) and adjusting the arrangement angle of the wavelength-selective reflector according to the sensed amount of light (S40). In addition, if necessary, the path of transmitted light may be additionally adjusted (S50). Through these operations, light may be obtained with desired optical output level.

In the drawing, an example is illustrated in which, for output adjustment, light reflected by the wavelength-selective reflector is sensed, and the arrangement angle of the wavelength-selective reflector is adjusted by using results of the sensing. However, this is a non-limiting example. For example, it is also possible to sense light passing through the wavelength-selective reflector and adjust the arrangement angle of the wavelength-selective reflector by using results of the sensing.

The above-described concept may be implemented in various devices, and implemented example structures will now be described.

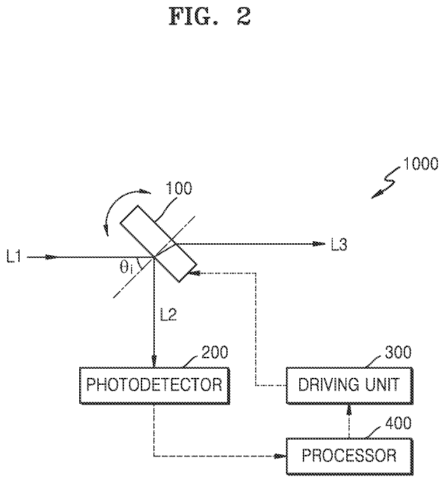

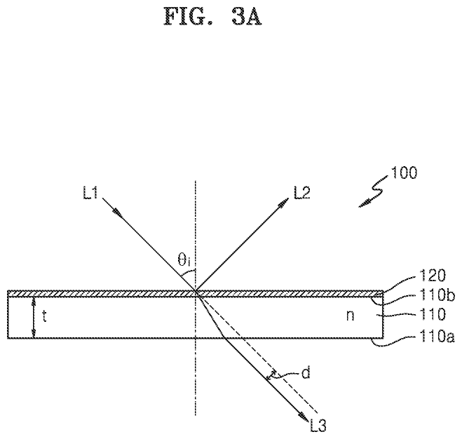

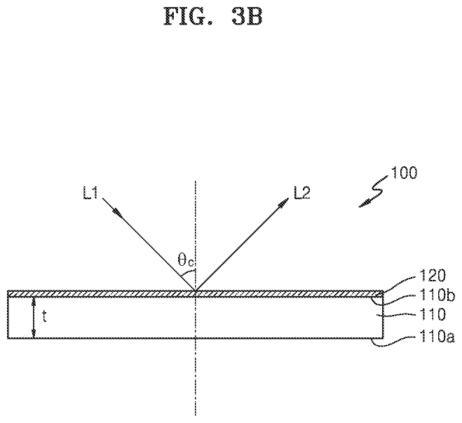

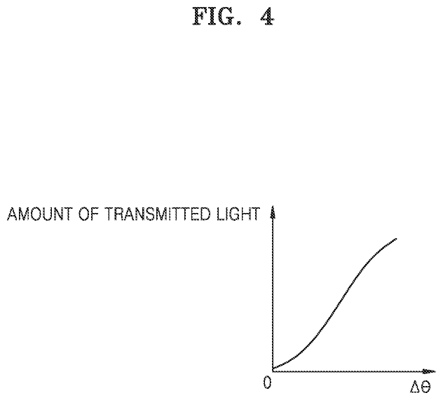

FIG. 2 illustrates a schematic structure of an optical output control device according to an embodiment. FIGS. 3A and 3B are views illustrating a detailed structure of a wavelength-selective reflector of the optical output control device shown in FIG. 2 and an optical path in which the amount of light transmission varies according to the angle of incidence of light. FIG. 4 is a graph illustrating an example in which the amount of light transmission varies according to the angle of incidence on the wavelength-selective reflector of the optical output control device shown in FIG. 2.

Referring to FIG. 2, an optical output control device 1000 includes: a wavelength-selective reflector 100 arranged such that a first light beam L1 is incident on the wavelength-selective reflector 100 at an incident angle θᵢ; and a driving unit 300 configured to rotate the wavelength-selective reflector 100 to adjust the incident angle θᵢ of light incident on the wavelength-selective reflector 100.

The optical output control device 1000 may further include: a photodetector 200 configured to sense the amount of light that passed through the wavelength-selective reflector 100; and a processor 400 configured to generate a control signal to be transmitted to the driving unit 300 based on the amount of light sensed by the photodetector 200.

The wavelength-selective reflector 100 is configured to reflect light in a predetermined wavelength band and incident at a set reference angle θ_{c}.

As shown in FIGS. 3A and 3B, the wavelength-selective reflector 100 includes: a transparent member 110 having an entrance surface 110b and an exit surface 110a which are opposite each other; and a wavelength-selective coating layer 120 formed on the entrance surface 110b and reflecting light in the predetermined wavelength band. In addition, an anti-reflection coating layer (not shown) may be formed on the exit surface 110a.

The wavelength-selective coating layer 120 may include a plurality of material layers having different optical properties. Incident light satisfying predetermined conditions may be totally reflected as a result of interaction between the incident light and the plurality of material layers. For example, light incident on an interface between media having different refractive indexes is reflected and transmitted while being refracted, and the total amount of reflected light and the total amount of transmitted light are determined by interference between the reflected light and the transmitted light which travel along a plurality of paths formed by a plurality of interfaces. Considering this interference, the refractive index and the thickness of each layer may be determined such that light satisfying specific incident angle conditions may be totally reflected. In addition, based on that optical properties depend on the wavelength of incident light, specific properties of the plurality of material layers of the wavelength-selective coating layer 120 may be set such that the wavelength-selective coating layer 120 may total reflect light having the predetermined wavelength band and incident at the set reference angle θ_{c}. The wavelength-selective coating layer 120, which is set as described above, reflects only a portion of light and transmits the other portion of the light, among light incident at an incident angle different from the set reference angle θ_{c}.

Referring to FIG. 3A, when a first light beam L1 having the predetermined wavelength band is incident on the wavelength-selective reflector 100 at the reference angle θ_{c}, all the incident first light beam L1 is reflected. That is, the amount of a second light beam L2, which is a reflected light beam, is equal to the amount of the first light beam L1.

As shown in FIG. 3B, when a first light beam L1 is incident on the wavelength-selective reflector 100 at an incident angle θᵢ different from the reference angle θ_{c}, a portion of the light is reflected, and the other portion of the light is transmitted. That is, the path of the first light beam L1 is divided into two paths: a path of a second light beam L2 which is a reflected light beam; and a path of to third light beam L3 which is a transmitted light beam. The amount of the third light beam L3 may vary according to the difference between the incident angle θᵢ and the reference angle θ_{c}.

In addition, due to the thickness (t) and the refractive index (n) of the transparent member 110, the direction of the third light beam L3 is different from the traveling direction in which the first light beam L1 is incident on the wavelength-selective reflector 100. The first light beam L1 is transmitted while being refracted at the entrance surface 110b and the exit surface 110a of the transparent member 110, and then travels along a path shifted by a distance (d) from the original traveling path thereof. As illustrated, when the entrance surface 110b and the exit surface 110a are parallel to each other, the traveling path of light is moved in parallel by the distance (d). However, this is a non-limiting example. For example, the entrance surface 110b and the exit surface 110a may not be parallel to each other, and in this case, the new path may not be parallel to the original path. A path change caused by the wavelength-selective reflector 100 may be adjusted by using an additional path-adjustment optical member, and this will be described later in other embodiments.

As illustrated in FIG. 4, the amount of transmitted light increases as Δθ defined by |θᵢ−θ_{c}| increases. The illustrated graph is an example, and the graph may be a straight line or another non-linear curve. The shape of the graph may vary depending on the detailed properties of the wavelength-selective coating layer 120 formed on the wavelength-selective reflector 100.

According to the properties of the wavelength-selective reflector 100, desired output may be obtained from the wavelength-selective reflector 100 by sensing the amount of light traveling via the wavelength-selective reflector 100 and adjusting the arrangement angle of the wavelength-selective reflector 100. To this end, the photodetector 200 may be used to sense light traveling via the wavelength-selective reflector 100, that is, to sense light reflected by or passing through the wavelength-selective reflector 100.

In FIG. 2, the photodetector 200 is arranged on a path of light reflected by the wavelength-selective reflector 100, that is, the photodetector 200 may sense the amount of light reflected by the wavelength-selective reflector 100.

The processor 400 may set the rotation angle of the wavelength-selective reflector 100 based on the amount of light sensed by the photodetector 200. For example, the processor 400 may predict an output value of the wavelength-selective reflector 100 from the amount of light sensed by the photodetector 200. That is, the processor 400 may predict the amount of a third light beam L3 passing through the wavelength-selective reflector 100. Then, the processor 400 may calculate a rotation angle for the wavelength-selective reflector 100 by comparing the predicted output value with a set target value.

The driving unit 300 may rotate the wavelength-selective reflector 100 under the control of the processor 400. The axis of rotation, on which the wavelength-selective reflector 100 is rotated, is perpendicular to a plane determined by the path of a first light beam L1 which is incident light, the optical path of a second light beam L2 which is reflected light, and the path of a third light beam L3 which is transmitted light, and according to the rotation of the wavelength-selective reflector 100 on the axis of rotation, the incident angle θᵢ of the first light beam L1 incident on the wavelength-selective reflector 100 is varied. A variation in the incident angle θᵢ results in a variation in the amount of transmitted light L3. That is, desired output may be obtained by appropriately adjusting the rotation angle of the wavelength-selective reflector 100.

The optical output control method described above is different from optical output control methods of the related art which use a specific polarized state of light. In optical output control methods of the related art, input light is polarized light, and thus, there may be spatial regions having slightly different polarization components. In this case, the distribution of an output-controlled beam is not uniform.

The optical output control device of the embodiment may be used regardless of the polarization state of light, and thus has substantially no problem regarding non-uniformity of output-controlled light.

Hereinafter, examples of optical output devices will be described according to various embodiments.

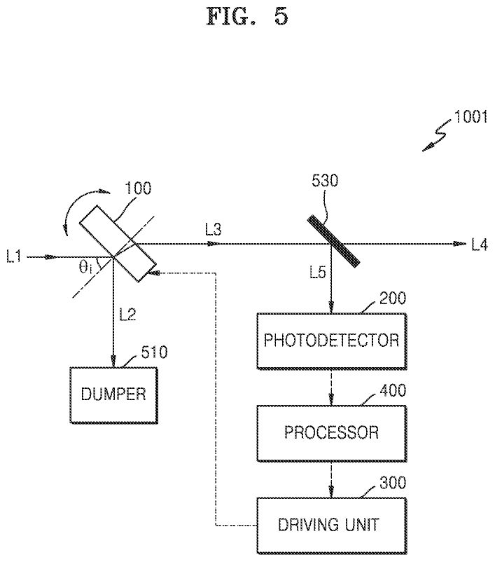

FIG. 5 illustrates a schematic structure of an optical output control device according to another embodiment.

An optical output control device 1100 of the current embodiment is different from the optical output control device 1000 shown in FIG. 2 in that a dumper 510 is arranged on the path of a second light beam L2 reflected by a wavelength-selective reflector 100, and a photodetector 200 is arranged at a different position.

The dumper 510 may be arranged on a reflection path of the wavelength-selective reflector 100 to simply process a reflected light beam, and the photodetector 200 may be arranged to detect the amount of light branching off from a third light beam L3 that has passed through the wavelength-selective reflector 100. To this end, a half mirror 530 may be arranged on a path of the third light beam L3 that passed through the wavelength-selective reflector 100. The half mirror 530 is a member configured to transmit half of incident light and reflect the rest of the incident light. The output value of a fourth light beam L4 passing through the half mirror 530 may be predicted by detecting the amount of a fifth light beam L5 reflected by the half mirror 530. Based on this, the wavelength-selective reflector 100 may be rotated to increase or decrease the output value to a desired value, thereby obtaining a desired output value. In the description of the current embodiment, the arrangement of the half mirror 530 is an example, and another type of beam splitter configured to split light may be used instead of the half mirror 530.

In the following embodiments, the photodetector 200 is illustrated as being arranged on a path of light reflected from the wavelength-selective reflector 100, but embodiments are not limited thereto. That is, the following embodiments may be modified like the current embodiment such that the photodetector 200 may be arranged on a path of light that passed through the wavelength-selective reflector 100.

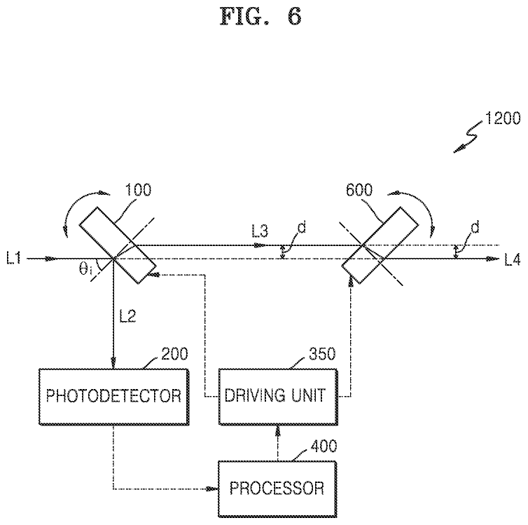

FIG. 6 illustrates a schematic structure of an optical output control device according to another example embodiment.

An optical output control device 1200 according to the present embodiment is different from the optical output control device 1000 shown in FIG. 2, in that the optical output control device 1200 further includes an optical path adjuster 600 arranged on a path of light passing through a wavelength-selective reflector 100.

As described with reference to FIG. 3B, the path of a third light beam L3 that has passed through the wavelength-selective reflector 100 may be shifted by a distance (d) from the path of a first light beam L1 which is an incident light beam. The optical path adjuster 600 may be rotated to adjust the path of light that passed through the wavelength-selective reflector 100 to be aligned with the path of light incident on the wavelength-selective reflector 100.

Based on the amount of light sensed by a photodetector 200, a processor 400 may generate a driving signal for rotating the wavelength-selective reflector 100 and also a driving signal for rotating the optical path adjuster 600.

A driving unit 350 may be configured to independently drive the wavelength-selective reflector 100 and the optical path adjuster 600. The optical path adjuster 600 may be driven to rotate to correct a deviation of the path of light which is caused by the wavelength-selective reflector 100, or in addition to this, the optical path adjuster 600 may be driven to rotate so that another path change is possible. In this case, a driving force independent of rotation of the wavelength-selective reflector 100 may be transmitted to the optical path adjuster 600.

Alternatively, the driving unit 350 may include, for example, a driving force transmitting unit such as a gear configured to transmit a driving force generated from a single driving source to both the wavelength-selective reflector 100 and the optical path adjuster 600. The driving force transmitting unit may be specifically set to transmit a driving force to the optical path adjuster 600 for correcting an optical path deviation caused by the rotation of the wavelength-selective reflector 100. For example, a driving force generated from the single driving source may be transmitted to the wavelength-selective reflector 100 and the optical path adjuster 600 according to a predetermined relationship.

As shown in the drawing, the optical path adjuster 600 may be arranged symmetrically to the wavelength-selective reflector 100 with respect to a plane which is perpendicular to the traveling path of light passing through the wavelength-selective reflector 100. In addition, the optical path adjuster 600 may include a material having the same refractive index as that of a transparent member of the wavelength-selective reflector 100 and may have the same thickness as the transparent member. An anti-reflection coating layer (not shown) may be formed on an entrance surface and/or an exit surface of the light path controller 600.

When the optical path adjuster 600 includes a material having the same refractive index as that of the transparent member of the wavelength-selective reflector 100 and has the same thickness of the wavelength-selective reflector 100, an optical path deviation caused by the wavelength-selective reflector 100 may be corrected by rotating the optical path adjuster 600 by the same angle as the wavelength-selective reflector 100 in the opposite direction to the direction in which the wavelength-selective reflector 100 is rotated. However, this is a non-limiting example, and the optical path adjuster 600 may have another thickness and another refractive index. In this case, the optical path adjuster 600 may be rotated to a different angle to correct an optical path deviation.

The driving unit 350 may be configured to drive the optical path adjuster 600 according to the rotation of the wavelength-selective reflector 100 such that the optical path adjuster 600 may be rotated the same angle as the wavelength-selective reflector 100 in the opposite direction to the direction in which the wavelength-selective reflector 100 is rotated. For example, the driving force transmitting unit may be configured such that a driving force generated from the single driving source may be transmitted to both the wavelength-selective reflector 100 and the optical path adjuster 600 with the same magnitude but in opposite directions.

The optical output devices 1000, 1100, and 1200 described above may be applied to various optical devices requiring output control.

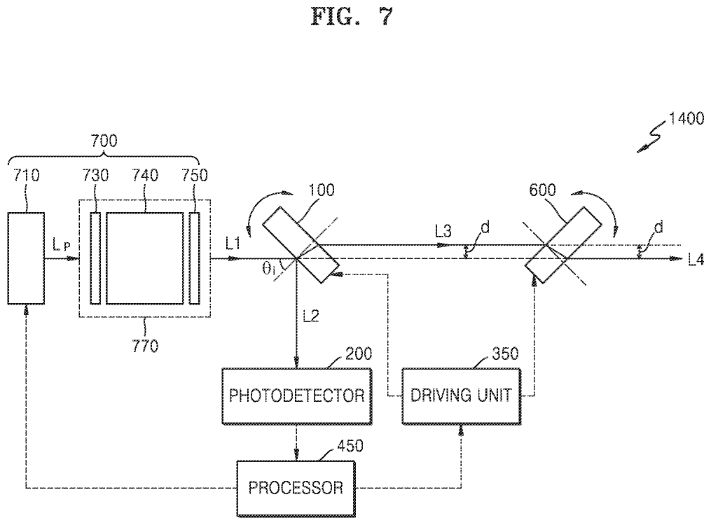

FIG. 7 is a view illustrating a schematic structure of a laser device according to an embodiment.

A laser device 1400 includes: a light source unit 700 configured to generate and output laser light in a predetermined wavelength band; a wavelength-selective reflector 100 arranged on a path of light emitted from the light source unit 700 and configured to reflect light having the predetermined wavelength band and incident at a reference angle θ_{c}; a photodetector 200 configured to sense the amount of light passing through the wavelength-selective reflector 100; a driving unit 350 configured to rotate the wavelength-selective reflector 100 for adjusting the angle of incidence of light incident on the wavelength-selective reflector 100; and a processor 450 configured to generate a control signal to be transmitted to the driving unit 350.

The light source unit 700 may include a laser medium 740, an excitation light source 710 configured to supply light to the laser medium 740, and a first mirror 730 and a second mirror 750 which are arranged with the laser medium 740 therebetween. The first mirror 730, the laser medium 740, and the second mirror 750 constitute a laser oscillation unit 770 in which light from the excitation light source 710 oscillates to produce laser light.

The excitation light source 710, which may be a flash lamp, emits light by receiving power from a power supply (not shown) and provides the light to the laser medium 740. The excitation light source 710 is not limited to a flash lamp and may include a laser diode.

The laser medium 740 absorbs the energy of light supplied from the excitation light source 710 and emits amplified light. The laser medium 740 may be neodymium-doped yttrium aluminum garnet (Nd:Yag). However, the laser medium 740 is not limited thereto, and Er:Yag may be used as the laser medium 135.

The first mirror 730 and the second mirror 750 may be arranged facing each other with the laser medium 740 therebetween to form a resonance path of light amplified by the laser medium 740. The reflectivity of the first and second mirrors 730 and 750 may be set such that the first mirror 730 may function as a reflection mirror, and the second mirror 750 may function as an output mirror.

The elements and arrangement of the light source unit 700 are described as an example of a basic structure for generating laser light. That is, the light source unit may include additional optical elements for controlling the properties of laser light to be emitted, and the arrangement of optical elements of the light source unit 700 may be modified.

A first light beam L1, which is laser light generated from the light source unit 700, may be in a specific polarization state or a non-polarized state. Because the laser device 1400 of the embodiment is capable of adjusting optical output regardless of the polarization state of light, the laser device 1400 does not require additional optical elements such as a polarizer or a phase retarder, which are used in laser devices of the related art to polarize light for optical output control.

The wavelength-selective reflector 100 may totally reflect a first laser beam L1 generated from the light source unit 700 in a predetermined wavelength band when the first laser beam L1 is incident on the wavelength-selective reflector 100 at the reference angle θ_{c}. The wavelength-selective reflector 100 may be specifically configured as illustrated in FIGS. 3A and 3B, and a wavelength-selective coating layer provided on the wavelength-selective reflector 100 may be set to match the wavelength band of light generated from the light source unit 700.

For example, the light source unit 700 may generate light in a wavelength of about 1064 nm, and the wavelength-selective reflector 100 may have a wavelength-selective coating layer that totally reflects light in a wavelength of 1064 nm and incident at an incident angle of 45 degrees. However, this is merely a non-limiting example.

The wavelength-selective reflector 100 may be oriented such that the incident angle θᵢ of a first light beam L1 generated from the light source unit 700 is different from the reference angle θ_{c}. In this case, the first light beam L1 is split into a second light beam L2 as being reflected by the wavelength-selective reflector 100 and a third light beam L3 as being transmitted through the wavelength-selective reflector 100.

The photodetector 200 may be arranged on a path of the second light beam L2 to sense the amount of light reflected by the wavelength-selective reflector 100.

The processor 450 generates a control signal to be transmitted to the driver 350 based on the amount of light sensed by the photodetector 200. The processor 400 may also control the overall operation of the laser device 1400 including the driving of the excitation light source 710.

The laser device 1400 may further include an optical path adjuster 600 arranged on a traveling path of the third light beam L3 transmitted through the wavelength-selective reflector 100, and the optical path adjuster 600 is configured to be rotated for controlling the traveling path.

The optical path adjuster 600 adjusts the traveling path of the first light beam L1 coming from the light source unit 700 such that the traveling path of the first light beam L1 after the first light beam L1 passes through the wavelength-selective reflector 100 may be aligned with the traveling path of the first light beam L1 incident on the wavelength-selective reflector 100.

The optical path adjuster 600 may be arranged symmetrically to the wavelength-selective reflector 100 with respect to a plane perpendicular to the traveling path of light that passed through the wavelength-selective reflector 100. In addition, the optical path adjuster 600 may include a material having the same refractive index as that of a transparent member of the wavelength-selective reflector 100 and may have the same thickness as the transparent member.

The driving unit 350 may drive the optical path adjuster 600 according to the rotation of the wavelength-selective reflector 100 such that the optical path adjuster 600 may be rotated the same angle as the wavelength-selective reflector 100 in the opposite direction to the rotation direction of the wavelength-selective reflector 100.

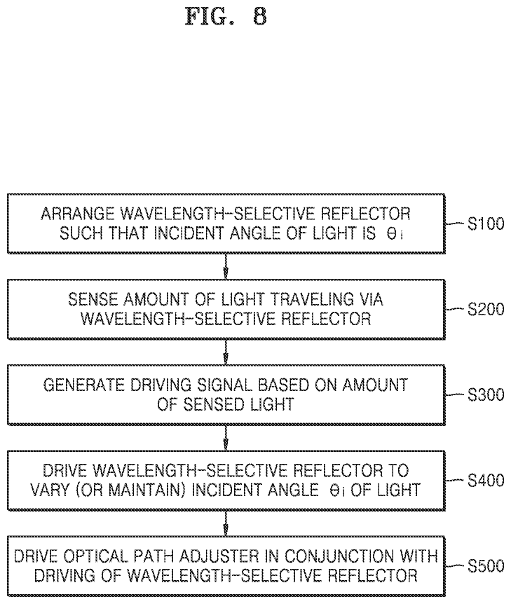

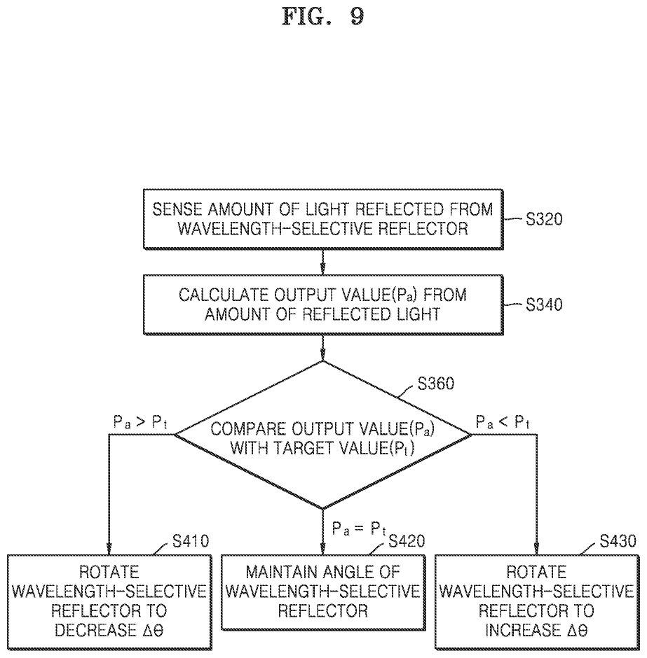

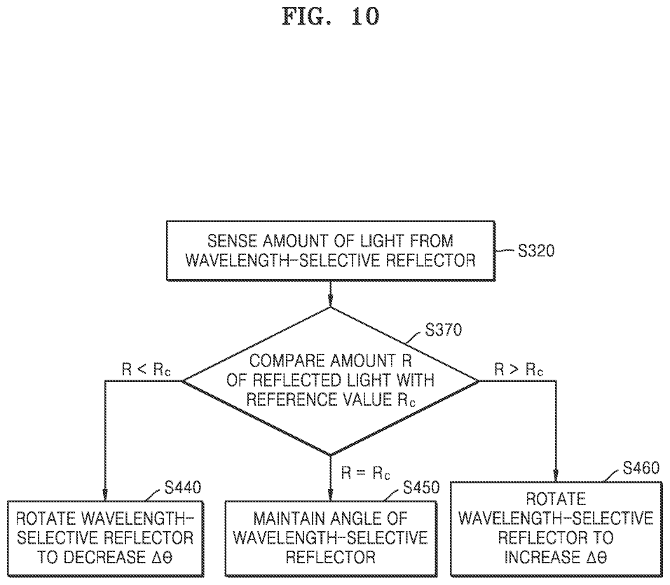

FIG. 8 is a flowchart conceptually illustrating operations in which the laser device shown in FIG. 7 emits controlled output light, and FIGS. 9 and 10 are example flowcharts exemplarily illustrating more detailed operations of controlling output light in the laser device shown in FIG. 7.

Referring to FIG. 8, the wavelength-selective reflector 100 is arranged such that the incident angle of light may be θᵢ (S100). In this case, the incident angle θᵢ may be different from the reference angle θ_{c}. Light incident on the wavelength-selective reflector 100 is transmitted and reflected, and the reflected light or transmitted light is sensed by the photodetector 200 (S200). Next, a driving signal to be transmitted to the driving unit 350 is generated based on the amount of sensed light (S300). The wavelength-selective reflector 100 is driven according to the driving signal such that the incident angle θᵢ may be changed or maintained (S400). In addition, the optical path adjuster 600 is driven in conjunction with the driving of the wavelength-selective reflector 200 (S500).

As illustrated in FIG. 9, an operation in which the processor 450 generates a driving signal may be performed based on a target value to be output. The photodetector 200 may sense the amount of light reflected from the wavelength-selective reflector 100 (S320), and the processor 450 may predict an output value Pₐ from the sensed amount of reflected light (S340). Next, the predicted output value Pₐ is compared with a set target value Pt (S360), and the rotation angle of the wavelength-selective reflector 100 is set according to results of the comparison.

When results of the comparison between the output value Pₐ and the target value Pt shows that the output value Pₐ is greater than the target value Pt, the wavelength-selective reflector is rotated in a direction for obtaining a lower output value (S410). That is, the processor 450 generates a driving signal for rotating the wavelength-selective reflector 100 in a direction decreasing the difference Δθ between the reference angle θ_{c} and the incident angle θᵢ at which light is incident on the wavelength-selective reflector 100, and the driving unit 350 rotates the wavelength-selective reflector 100 according to the driving signal.

When the output value Pₐ is less than the target value Pt, the wavelength-selective reflector 100 is rotated in a direction for obtaining a higher output value (S430). That is, the processor 450 generates a driving signal for rotating the wavelength-selective reflector 100 in a direction increasing the difference Δθ between the reference angle θ_{c} and the incident angle θᵢ at which light is incident on the wavelength-selective reflector 100, and the driving unit 350 rotates the wavelength-selective reflector 100 according to the driving signal.

When the output value Pₐ is equal to the target value Pt, the arrangement angle of the wavelength-selective reflector 100 is maintained (S420). That is, because the difference Δθ in the current state corresponds to the target value Pt, the wavelength-selective reflector 100 is not rotated to maintain the incident angle θᵢ corresponding to the difference Δθ.

Alternatively, as illustrated in FIG. 10, the operation in which the processor 450 generates a driving signal may be performed by setting a reference value and comparing the reference value with the amount of light reflected by the wavelength-selective reflector 100 to obtain a target output value.

The photodetector 200 senses the amount of light reflected from the wavelength-selective reflector 100 (S320), and the processor 450 compares the sensed amount of reflected light with a set reference value R_{c} (S370). Then, a rotation angle is set for the wavelength-selective reflector 100 according to results of the comparison.

When the amount R of reflected light sensed by the photodetector 200 is less than the set reference value R_{c}, the wavelength-selective reflector 100 is rotated in a direction increasing the amount of reflected light. That is, the processor 450 generates a driving signal for rotating the wavelength-selective reflector 100 in a direction decreasing the difference Δθ between the reference angle θ_{c} and the incident angle θᵢ at which light is incident on the wavelength-selective reflector 100, and the driving unit 350 rotates the wavelength-selective reflector 100 according to the driving signal (S440).

When the amount R of reflected light sensed by the photodetector 200 is greater than the set reference value R_{c}, the wavelength-selective reflector 100 is rotated in a direction decreasing the amount of reflected light. The processor 450 generates a driving signal for rotating the wavelength-selective reflector 100 in a direction increasing the difference Δθ between the reference angle θ_{c} and the incident angle θᵢ at which light is incident on the wavelength-selective reflector 100, and the driving unit 350 rotates the wavelength-selective reflector 100 according to the driving signal (S460).

When the amount R of reflected light sensed by the photodetector 200 is equal to the set reference value R_{c}, the arrangement angle of the wavelength-selective reflector 100 is maintained (S450). Because the difference Δθ in the current state corresponds to the target value Pt, the wavelength-selective reflector 100 is not rotated to maintain the incident angle θᵢ corresponding to the difference Δθ.

According to the configuration and operations described above, the laser device 1400 of the embodiment may adjust its output and maintain a uniform output distribution regardless of the polarization state of laser light generated from the light source unit 700.

Various embodiments of the present disclosure have been described in detail, and those of ordinary skill in the art to which the present disclosure pertains may make various modifications therein without departing from the spirit and scope of the present disclosure as defined by the appended claims. Therefore, such modifications should be construed as being included within the scope of the present disclosure.

1. An optical output control device comprising:
a wavelength-selective reflector configured to reflect light in a predetermined wavelength band and incident at a reference angle θ_{c}; and
a driving unit configured to rotate the wavelength-selective reflector to adjust an angle of incidence of light incident on the wavelength-selective reflector.

2. The optical output control device of claim 1, further comprising:
a photodetector configured to sense an amount of light reflected from the wavelength-selective reflector or passing through the wavelength-selective reflector; and
a processor configured to generate, based on the amount of light sensed by the photodetector, a control signal to be transmitted to the driving unit.

3. The optical output control device of claim 2, wherein the processor is further configured to predict an output value from the amount of sensed light, and calculate a rotation angle for the wavelength-selective reflector by comparing the predicted output value with a set target value.

4. The optical output control device of claim 1, wherein the wavelength-selective reflector comprises:
a transparent member having an entrance surface and an exit surface, which are opposite each other; and
a wavelength-selective coating layer formed on the entrance surface to reflect light in the predetermined wavelength band.

5. The optical output control device of claim 4, wherein an anti-reflection coating layer is formed on the exit surface.

6. The optical output control device of claim 1, further comprising an optical path adjuster arranged on a traveling path of light that passed through the wavelength-selective reflector and configured to be rotated for adjusting the traveling path.

7. The optical output control device of claim 6, wherein the optical path adjuster is further configured to adjust the traveling path of light that passed through the wavelength-selective reflector to be aligned with a traveling path of the light when the light is incident on the wavelength-selective reflector.

8. An optical device comprising the optical output control device of claim 1.

9. A laser device comprising:
a light source unit comprising a laser medium, a first mirror and a second mirror which are arranged with the laser medium therebetween, and an excitation light source configured to supply light to the laser medium, the light source unit being configured to generate light in a predetermined wavelength band;
a wavelength-selective reflector arranged on a path of light output from the light source unit and configured to reflect light in the predetermined wavelength band and incident at a reference angle θ_{c};
a photodetector configured to sense an amount of light reflected from the wavelength-selective reflector or passing through the wavelength-selective reflector;
a driving unit configured to rotate the wavelength-selective reflector to adjust an angle of incidence of light incident on the wavelength-selective reflector; and
a processor configured to generate a control signal to be transmitted to the driving unit.

10. The laser device of claim 9, wherein the wavelength-selective reflector is arranged such that an incident angle θᵢ at which light generated from the light source unit is incident is different from the reference angle θ_{c}.

11. The laser device of claim 9, wherein the processor is further configured to generate the control signal based on the amount of light sensed by the photodetector.

12. The laser device of claim 11, wherein the photodetector is arranged to sense the amount of light reflected from the wavelength-selective reflector.

13. The laser device of claim 12, wherein the processor is further configured to predict an output value from the amount of reflected light, and calculate a rotation angle for the wavelength-selective reflector by comparing the predicted output value with a set target value.

14. The laser device of claim 12, wherein the processor is further configured to calculate a rotation angle for the wavelength-selective reflector by comparing the amount of reflected light with a set reference value.

15. The laser device of claim 9, wherein the wavelength-selective reflector comprises:
a transparent member having an entrance surface and an exit surface, which are opposite each other; and
a wavelength-selective coating layer formed on the entrance surface to reflect light in the predetermined wavelength band.

16. The laser device of claim 15, further comprising an optical path adjuster arranged on a traveling path of light that passed through the wavelength-selective reflector and configured to be rotated for adjusting the traveling path.

17. The laser device of claim 16, wherein the optical path adjuster is further configured to adjust the traveling path of light that passed through the wavelength-selective reflector to be aligned with a traveling path of the light when the light is incident on the wavelength-selective reflector.

18. The laser device of claim 16, wherein the optical path adjuster is arranged symmetrically to the wavelength-selective reflector with respect to a plane perpendicular to the traveling path of light that passed through the wavelength-selective reflector.

19. The laser device of claim 16, wherein the optical path adjuster comprises a material having a refractive index equal to a refractive index of the transparent member and has a thickness equal to a thickness of the transparent member.

20. The laser device of claim 16, wherein the driving unit is further configured to rotate the optical path adjuster in conjunction with rotation of the wavelength-selective reflector such that the optical path adjuster is rotated by a rotation angle of the wavelength-selective reflector in a direction opposite to a rotation direction of the wavelength-selective reflector.
